(19) United States
(12) Patent Application Publication
(10) Pub. No.: US 2011/0189293 A1
(43) Pub. Date: Aug. 4, 2011

(54) THERAPEUTIC REGIMENS FOR THE TREATMENT OF IMMUNOINFLAMMATORY DISORDERS

(75) Inventor: Mahesh V. Padval, Waltham, MA (US)

(73) Assignee: CombinatoRx, Incoporated, Cambridge, MA (US)

(21) Appl. No.: 12/808,477

(22) PCT Filed: Dec. 17, 2008
(86) PCT No.: PCT/US08/13805

§ 371 Date: Apr. 14, 2011

Related U.S. Application Data
(60) Provisional application No. 61/014,307, filed on Dec. 17, 2007.

(51) Int. Cl. A61K 9/14; A61K 31/519; A61K 31/56

(52) U.S. Cl. 424/489; 514/262.1; 514/171; 514/170

(57) ABSTRACT

A method for treating an immunoinflammatory disorder in a subject in need thereof, said method comprising administering to said subject a unit dosage form comprising dipyridamole coated onto acid beads and formulated for controlled release. The method further including administering a corticosteriod concurrently with administration of the dipyridamole.

### BACKGROUND OF THE INVENTION

The combination of prednisolone and dipyridamole is an orally available synergistic drug candidate in Phase 2 clinical development for the treatment of immunoinflammatory disorders. A synergistic drug includes two compounds that are designed to act synergistically through multiple pathways to provide a therapeutic effect which neither component administered alone and at the same dosing levels can achieve. The combination of prednisolone with dipyridamole was designed to selectively amplify certain elements of prednisolone's anti-inflammatory and immunomodulatory activities, without replicating steroid side effects.

Proper formulation is essential to maximize the therapeutic benefit of a synergistic drug combination.

### SUMMARY OF THE INVENTION

In one aspect, the invention features a method for treating an immunoinflammatory disorder in a subject in need thereof, the method including administering (e.g., once, twice, or three times a day) to the subject a unit dosage form including dipyridamole coated onto acid beads and formulated for controlled release. The unit dosage form can include between 40 and 400 mg of dipyridamole (e.g., 45 mg, 90 mg, 180 mg, or 360 mg). In some embodiments, the dipyridamole is coated onto tartaric acid beads in a wt/wt (dipyridamole:tartaric acid) ratio of, for example, 1:0.8, 1:0.6, 1:07, 1:0.9, 1:1, 1:1.1, or 1:1.2.

In certain embodiments, the dipyridamole can be coated with a controlled release coating (e.g., hydroxypropyl methylcellulose phthalate 55, Surelease®:HPMC E5, or Eudragit® L100:Eudragit® S100).

In still other embodiments, the unit dosage form includes dipyridamole formulated for immediate release. The percentage of dipyridamole formulated for controlled release can be between 20% and 100% (e.g., from 50% to 80%, 55% to 85%, 60% to 90%, 65% to 95%, 45% to 75%, 45% to 55%, 50% to 60%, 55% to 65%, 70% to 80%, 75% to 85%, 80% to 90%, or 85% to 95%) of the dipyridamole in the unit dosage form.

In another embodiment, the method further includes administering to the subject a corticosteroid (e.g., prednisolone, prednisone, budesonide, methylprednisolone, fluticasone, betamethasone, or deflazacort). The corticosteroid can be administered in two separate doses. For example, the first dose can be administered (e.g., at waking) in a unit dosage formulation including from 0.75 to 3.75 mg (e.g., 1.5 to 2.5 mg, 0.75 to 2.0 mg, 2.0 mg to 3.75 mg, 0.9 mg, or 1.8 mg) of prednisolone or an equivalent, equipotent amount of another corticosteroid, and the second dose is administered within 8 hours of the first dose (e.g., 4-6, 3-5, or 2-4 hours after the first dose) in a unit dosage formulation including from 0.75 to 3.75 mg (e.g., 0.75 to 1.25, 1.5 to 2.5 mg, 0.75 to 2.0 mg, 2.0 mg to 3.75 mg, 0.9 mg, or 1.8 mg) of prednisolone or an equivalent, equipotent amount of another corticosteroid. The first and second dose of corticosteroid can be formulated for either immediate or controlled release, the first dose can be formulated for immediate release and the second dose for controlled release, or the first dose can be formulated for controlled release and the second dose for immediate release. In one particular embodiment, the first dose is administered in a unit dosage formulation including from 1.0 to 2.5 mg of prednisolone or an equivalent, equipotent amount of another corticosteroid, formulated for immediate release and the second dose is administered in a unit dosage formulation including from 0.75 to 2.0 mg of prednisolone or an equivalent, equipotent amount of another corticosteroid, formulated for controlled release.

In a related aspect, the invention features a pharmaceutical composition in unit dosage form including dipyridamole coated onto tartaric acid beads and formulated for controlled release. The unit dosage form can include between 40 and 400 mg of dipyridamole (e.g., 45 mg, 90 mg, 180 mg, or 360 mg). In some embodiments, the dipyridamole is coated onto tartaric acid beads in a wt/wt (dipyridamole:tartaric acid) ratio of, for example, 1:0.8, 1:0.6, 1:07, 1:0.9, 1:1, 1:1.1, or 1:1.2.

In certain embodiments, the dipyridamole can be coated with a controlled release coating (e.g., hydroxypropyl methylcellulose phthalate 55, Surelease®:HPMC E5, or Eudragit® L100:Eudragit® S100).

In still other embodiments, the unit dosage form includes dipyridamole formulated for immediate release. The percentage of dipyridamole formulated for controlled release can be between 20% and 100% (e.g., from 50% to 80%, 55% to 85%, 60% to 90%, 65% to 95%, 45% to 75%, 45% to 55%, 50% to 60%, 55% to 65%, 70% to 80%, 75% to 85%, 80% to 90%, or 85% to 100%) of the dipyridamole in the unit dosage form.

This unit dosage form may further include administering to the subject a corticosteroid (e.g., prednisolone, prednisone, budesonide, methylprednisolone, fluticasone, betamethasone, or deflazacort). The formulation of corticosteroid can be from 0.75 to 3.75 mg (e.g., 1.5 to 2.5 mg, 0.75 to 2.0 mg, 2.0 mg to 3.75 mg, 0.9 mg, or 1.8 mg) of prednisolone or an equivalent, equipotent amount of another corticosteroid. The corticosteroid can be formulated for controlled or immediate release, or a combination controlled release and immediate release. The percentage of corticosteroid formulated for controlled release can be between 20% and 100% (e.g., from 50% to 80%, 55% to 85%, 60% to 90%, 65% to 95%, 45% to 75%, 45% to 55%, 50% to 60%, 55% to 65%, 70% to 80%, 75% to 85%, 80% to 90%, or 85% to 100%). Corticosteroid formulated for controlled release can be formulated to release a substantial portion of the corticosteroid, for example, 2-8 hours, 4-6 hours, or 3-5 hours after administration. In one particular embodiment, the unit dosage form includes 0.75 to 3.75 mg of prednisolone, wherein 30% to 60%, 40% to 70%, 50% to 80%, or 60% to 90% of the prednisolone is formulated for immediate release and 10% to 40%, 20% to 50%, 30% to 60%, or 40% to 70% of the prednisolone is formulated for controlled release. In certain embodiments, the dipyridamole is coated onto an acid bead. In other embodiments, the dipyridamole is formulated as a homogenous bead.

In another aspect, the invention features a pharmaceutical composition in unit dosage form including 40 to 400 mg of dipyridamole (e.g., 45 mg, 90 mg, 180 mg, or 360 mg) formulated for controlled release, and 0.75 to 3.75 mg of prednisolone (e.g., 1.5 to 2.5 mg, 0.75 to 2.0 mg, 2.0 mg to 3.75 mg, 0.9 mg, or 1.8 mg) or an equivalent, equipotent amount of another corticosteroid, formulated for controlled release or immediate release.

In certain embodiments, the unit dosage form includes dipyridamole formulated for immediate release. The percentage of dipyridamole formulated for controlled release can be between 20% and 100% (e.g., from 50% to 80%, 55% to 85%, 60% to 90%, 65% to 95%, 45% to 75%, 45% to 55%, 50% to 60%, 55% to 65%, 70% to 80%, 75% to 85%, 80% to 90%, or 85% to 95%) of the dipyridamole in the unit dosage form.

In another embodiment, the unit dosage form includes a corticosteroid formulated for a combination controlled release and immediate release. The percentage of corticosteroid formulated for controlled release can be between 20% and 100% (e.g., from 50% to 80%, 60% to 80%, 30% to 60%, 40% to 70%, 45% to 75%, or 80% to 100%). Corticosteroid formulated for controlled release can be formulated to release a substantial portion of the corticosteroid, for example, 2-8 hours, 4-6 hours, or 3-5 hours after administration. In one particular embodiment, the unit dosage form includes 0.75 to 3.75 mg of prednisolone, wherein 30% to 60%, 40% to 70%, 50% to 80%, or 60% to 90% of the prednisolone is formulated for immediate release and 10% to 40%, 20% to 50%, 30% to 60%, or 40% to 70% of the prednisolone is formulated for controlled release.

In certain embodiments, the pharmaceutical composition of the invention includes an inner core including prednisolone formulated for controlled release and an outer coating including prednisolone formulated for immediate release. For example, the inner core can include from 0.75 to 1.25 mg (e.g., 0.75 to 1.1 mg, 0.65 to 1.1 mg, 0.80 mg to 1.0 mg, or 0.9 mg) of prednisolone formulated for controlled release and an outer coating comprising 1.25 to 2.25 mg (e.g., 1.5 to 2.0 mg, 1.6 to 2.0 mg, 1.7 mg to 2.0 mg, or 1.8 mg) of prednisolone formulated for immediate release. In other embodiments, the size of the pill is reduced and the dosing regimen increased by having the inner core include from 0.25 to 0.75 mg (e.g., 0.35 to 0.65 mg, 0.35 to 0.75 mg, 0.25 mg to 0.55 mg, or 0.45 mg) of prednisolone formulated for controlled release and an outer coating comprising 0.75 to 1.25 mg (e.g., 0.75 to 1.1 mg, 0.65 to 1.1 mg, 0.80 mg to 1.0 mg, or 0.9 mg) of prednisolone formulated for immediate release.

The invention also features a kit including any of the forgoing pharmaceutical compositions and instructions for administering (e.g., once, twice, or three times daily) the pharmaceutical composition for the treatment of an immunoinflammatory disease.

In an embodiment of any of the above methods, compositions, and kits, the pharmaceutical composition of the invention includes a corticosteroid formulated in a unit dosage form having a dissolution release profile under in vitro conditions in which at least 55%, 60%, 65%, 70%, or 75% of the corticosteroid is released within the first two hours of testing, wherein the in vitro conditions employ USP Dissolution Apparatus No. 1 at 37° C.±0.5° C. and 100 rpm in 0.1N HCl as dissolution medium for the first two hours, and a pH 6.8 phosphate buffer as the medium thereafter. Desirably, the corticosteroid formulated in a unit dosage has a dissolution release profile under in vitro conditions in which at least 50%, 55%, 60%, 65%, 70%, or 75% of the corticosteroid is released within the first 30 minutes, 45 minutes, or 60 minutes of testing, wherein the in vitro conditions employ USP Dissolution Apparatus No. 1 at 37° C.±0.5° C. and 100 rpm in 0.1N HCl as dissolution medium for the first two hours, and a pH 6.8 phosphate buffer as the medium thereafter.

In still another embodiment of any of the above methods, compositions, and kits, the pharmaceutical composition of the invention includes dipyridamole formulated in a unit dosage form having a dissolution release profile under in vitro conditions in which at least 10-55% (i.e., 15-55%, 20-55%, 25-55%, 25-45%, 35-55%, 30-45%, or 40-55%) of the dipyridamole is released within the first two hours of testing and not less than 80%, 82%, 84%, 86%, 88%, 90%, 91%, 93%, 95%, or 97% of the dipyridamole is released within 8 hours, wherein the in vitro conditions employ USP Dissolution Apparatus No. 1 at 37° C.±0.5° C. and 100 rpm in 0.1N HCl as dissolution medium for the first two hours, and a pH 6.8 phosphate buffer with 0.25% sodium lauryl sulfate as the medium thereafter.

In a further embodiment of any of the above methods, compositions, and kits, the pharmaceutical composition of the invention includes dipyridamole formulated in a unit dosage form having, upon administration to fed patients (normal breakfast), an absorption rate constant of from 0.20 to 0.40, 0.22 to 0.42, 0.24 to 0.44, 0.26 to 0.46, 0.28 to 0.48, 0.30 to 0.50, 0.32 to 0.52, 0.34 to 0.54, 0.36 to 0.56, 0.38 to 0.58, 0.40 to 0.60, 0.40 to 0.60, 0.42 to 0.62, 0.44 to 0.64, 0.46 to 0.66, 0.48 to 0.68, 0.50 to 0.70, 0.52 to 0.72, 0.54 to 0.74, 0.56 to 0.76, 0.58 to 0.78, 0.60 to 0.80, 0.62 to 0.82, 0.64 to 0.84, 0.66 to 0.86, 0.68 to 0.88, 0.70 to 0.90, 0.72 to 0.92, 0.74 to 0.94, 0.76 to 0.96, 0.78 to 0.98, 0.30 to 0.66, 0.33 to 0.69, 0.36 to 0.72, 0.39 to 0.75, 0.43 to 0.78, 0.46 to 0.80, 0.49 to 0.83, 0.52 to 0.86, or 0.55 to 0.89 l/hr.

The term “absorption rate constant” refers to the average absorption rate constant observed for dipyridamole in a pharmacokinetic study involving 12 or more subjects following a normal breakfast as described in Example 9. The absorption rate constant can be determined by measuring circulating concentrations of dipyridamole in each dosed subject following a meal and fitting the resulting data for each individual subject using commercially available algorithms as described in Example 9.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, antineutrophil cytoplasmic antibodies (ANCA) associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease (COPD); cirrhosis; Cogan's syndrome; contact dermatitis; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis;

hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea (e.g., caused by sarcoidosis, scleroderma, Sweet's syndrome, systemic lupus erythematosus, urticaria, zoster-associated pain, among others); sarcoidosis; scleroderma; segmental glomerulosclerosis; septic shock syndrome; shoulder tendonitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis.

By “corticosteroid” is meant any naturally occurring or synthetic steroid hormone which can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at A4, a C3 ketone, and a C20 ketone. Corticosteroids may have glucocorticoid and/or mineralocorticoid activity. In preferred embodiments, the corticosteroid is prednisolone. Exemplary corticosteroids are 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. Desirably, the corticosteroid is prednisolone.

By “acid bead” is meant a bead having an acid core that, when exposed to the gut, sufficiently lowers the local pH such that dipyridamole is soluble. Acid beads can include fumaric acid, malic acid, tartaric acid, citric acid, succinic acid, and/or ascorbic acid. In a preferred embodiment, the acid bead is a tartaric acid bead. Acid beads coated with dipyridmole are described in U.S. Pat. Nos. 4,361,546 and 4,367,217.

By “an effective amount” is meant the amount of a compound, in a combination of the invention, required to treat or prevent an immunoinflammatory disorder. The effective amount of active compound(s) used to practice the present invention for therapeutic treatment of conditions caused by or contributing to an inflammatory disease varies depending upon the manner of administration, the immunoinflammatory disorder being treated, the age, body weight, and general health of the patient. Ultimately, the attending physician or veterinarian will decide the appropriate amount and dosage regimen. Such amount is referred to as an effective amount.

By an “equivalent, equipotent amount” is meant a dosage of a corticosteroid that produces the same anti-inflammatory effect in a patient as a recited dosage of prednisolone.

By “immediate release” is meant that the therapeutically active component (e.g., a corticosteroid) is released from the formulation immediately such that 80%, 85%, 90%, or even 95% of the component in the formulation is absorbed into the blood stream of a patient less than two hours after oral administration. Whether a pharmaceutical composition is formulated for immediate release can be determined by measuring the pharmacokinetic profile of the formulation.

By “controlled release” is meant that the therapeutically active component is released from the formulation over a defined period of time, such that at a given dose, the Cₘₐₓ is decreased in comparison to the same dose of therapeutically active component formulated for immediate release. In controlled release formulations the Tₘₐₓ may or may not change.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “unit dosage form” and “unit dosage formulation” refer to physically discrete units suitable as unitary dosages, such as a pill, tablet, caplet, hard capsule, or soft capsule, each unit containing a predetermined quantity of dipyridamole and/or corticosteroid.

As used herein, the term “homogeneous bead” refers to a bead formulation including dipyridamole distributed throughout the bead along with other pharmaceutically acceptable excipients (e.g., diluents and binders). Homogeneous beads can be prepared as described in the examples.

As used herein, the term “coated” refers to a bead formulation including a corticosteroid, such as prednisolone, applied to the surface of a carrier, such as a non-pareil seed or tartaric acid bead. Coated beads can be prepared as described in the examples.

Other features and advantages of the invention will be apparent from the following detailed description, the drawings, and the claims.

### BRIEF DESCRIPTION OF THE DRAWINGS

FIG. 1 is a flow chart depicting the prednisolone bead manufacturing process.

FIG. 2 is a flow chart depicting the dipyridamole bead manufacturing process.

FIG. 3A and FIG. 3B are flow charts depicting the hydroxypropyl methylcellulose phthalate 55 coated dipyridamole bead manufacturing process.

FIG. 4A and FIG. 4B are flow charts depicting the Surelease®:HPMC E5 coated dipyridamole bead manufacturing process.

FIG. 5A and FIG. 5B are flow charts depicting the Eudragit® L100:Eudragit® S100 coated dipyridamole bead manufacturing process.

FIG. 6 is a flow chart depicting the dipyridamole/prednisolone capsule manufacturing process.

FIG. 7 is a graph showing percentage of drug release as a function of time of the indicated controlled release formulations (Variants B-D). These data show that differences in controlled release coating result in different drug release profiles.

FIG. 8 is a graph showing the in-vitro dissolution profile for dipyridamole from formulation variants D1 and D2.

FIG. 9 is a graph showing the in-vitro dissolution profile for prednisolone from formulation variants E and F.

### DETAILED DESCRIPTION

The invention provides for pharmaceutical compositions in unit dosage form containing dipyridamole, optionally with a corticosteroid. The compositions are useful, for example, for the treatment of immunoinflammatory disorders. Several formulations have been prepared and are described in the Examples (Example 1 (variant B), Example 2 (variant C), Example 3 (variant D), Example 4 (variant D0, Example 5 (variant D2), Example 6 (variant E), and Example 7 (variant F)).

### Corticosteroids

The combinations of the invention include a corticosteroid selected from the class of selective glucocorticosteroid receptor agonists (SEGRAs) including, without limitation, 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione, 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | | Schedule |
|---|---|---|---|---|---|
| Psoriasis | oral | Prednisolone | 7.5-60 | mg | per day or divided b.i.d. |
| | oral | Prednisone | 7.5-60 | mg | per day or divided b.i.d. |
| Asthma | inhaled | beclomethasone dipropionate | 42 | μg/puff) | 4-8 puffs b.i.d. |
| | inhaled | Budesonide | (200 | μg/inhalation) | 1-2 inhalations b.i.d. |
| | inhaled | Flunisolide | (250 | μg/puff) | 2-4 puffs b.i.d. |
| | inhaled | fluticasone propionate | (44, 110 or 220 | μg/puff) | 2-4 puffs b.i.d. |
| | inhaled | triamcinolone acetonide | (100 | μg/puff) | 2-4 puffs b.i.d. |
| COPD | oral | Prednisone | 30-40 | mg | per day |
| Crohn's disease | oral | Budesonide | 9 | mg | per day |
| Ulcerative colitis | oral | Prednisone | 40-60 | mg | per day |
| | oral | Hydrocortisone | 300 | mg (IV) | per day |
| | oral | Methylprednisolone | 40-60 | mg | per day |
| Rheumatoid arthritis | oral | Prednisone | 10 | mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone. Two or more corticosteroids can be administered in the same treatment.

Equivalent potency in clinical dosing is well known. Information relating to equivalent corticosteroid dosing may be found in the British National Formulary (BNF), 37 March 1999, the content of which is incorporated herein by reference.

The BNF guidelines are included in Table 2 below. More specifically, Table 2 provides doses of corticosteroids equivalent to 5 mg of prednisolone and equivalent to 1 mg of prednisolone when administered in a manner according to this invention.

TABLE 2
Equivalent Dose to Prednisolone

| | Equal to 5 mg | Equal to 1 mg |
|---|---|---|
| Drug | prednisolone | prednisolone |
| Betamethasone | 750 μg | 150 μg |
| cortisone acetate | 25 mg | 5 mg |
| Deflazacort | 6 mg | 1.2 mg |
| Dexamethasone | 750 μg | 150 μg |
| Hydrocortisone | 20 mg | 4 mg |
| methyl prednisone | 4 mg | 0.8 mg |
| Triamcinolone | 4 mg | 0.8 mg |

It is also known (BNF 37 March 1999) from clinical dosing equivalence that doses of triamcinolone, fluticasone, and budesonide are broadly similar in nasal administration (110 μg, 100 μg, and 200 μg).

Two or more corticosteroids can be administered in the same treatment, or present in the same kit or unit dosage formulation.

### Dipyridamole

The invention features unit dosage forms of dipyridamole of between 20 and 400 mg (e.g., 20, 30, 45, 90, 120, 180, 360, or 400 mg). These dosages can be formulated for controlled release (e.g., delayed release and sustained release) or immediate release using the methods and compositions described herein.

### Formulation

The combination of the invention may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients, preferably an excipient from the GRAS listing. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided in unit dosage form as chewable tablets, tablets, caplets, or capsules (e.g., as hard gelatin capsules wherein the active ingredient is mixed with an inert solid diluent, or as soft gelatin capsules wherein the active ingredient is mixed with water or an oil medium).

The formulations of the invention include diluents (e.g., lactose monohydrate, cellulose, glyceryl monostearate, and/or dibasic calcium phosphate, among others) and binders (e.g., polyvinylpyrrolidone, hypromellose, sucrose, guar gum, and/or starch). Any diluent or binder known in the art can be used in the methods, compositions, and kits of the invention.

The formulations of the invention may also include controlled release coatings. Such coatings include EUDRAGIT RL®, EUDRAGIT RS®, cellulose derivatives such as ethylcellulose aqueous dispersions (AQUACOAT®, SURELEASE®), hydroxyethyl cellulose, hydroxypropyl cellulose, hydroxypropyl methylcellulose, polyvinylpyrrolidone, polyvinylpyrrolidone/vinyl acetate copolymer, and OPADRY®.

### Kits

The individually or separately formulated agents of the invention can be packaged together, or individually, as a kit. Non-limiting examples include kits that contain, e.g., two pills, a pill and a capsule, a capsule containing multiple bead formulations, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like. Kits may also include instructions for administering the pharmaceutical compositions using any indication and/or dosing regimen described herein. Further description of kits is provided in the examples.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Drug Product

Dipyridamole and prednisolone were formulated in bead form and encapsulated in a standard size ‘0’ capsule. Six distinct capsule strengths were manufactured to accommodate the unequal amounts of prednisolone given in the morning and afternoon, and to allow for dose ranging. A dosing regimen including 1.8 mg prednisolone+180 mg dipyridamole administered at 0800 hours followed by 0.9 mg prednisolone+180 mg dipyridamole administered at 1300 hours has been shown to be efficacious in subjects with rheumatoid arthritis (RA) and osteoarthritis (OA). In this previous study both active ingredients were formulated for immediate release. The strengths are shown in Table 3.

TABLE 3
Prednisolone and Dipyridamole Quantities in Capsules

| | Prednisolone | Dipyridamole |
|---|---|---|
| Dosing Time | Quantity/Capsule | Quantity/Capsule |
| 0800 hours | 1.8 mg | 45 mg |
| | 1.8 mg | 90 mg |
| | 1.8 mg | 180 mg |
| 1300 hours | 0.9 mg | 45 mg |
| | 0.9 mg | 90 mg |
| | 0.9 mg | 180 mg |

The quantitative composition of the capsules is provided in Table 4 and Table 5, where the first table gives the quantitative compositions of the three dosage strengths that contain 0.9 mg prednisolone with varying amounts of dipyridamole and the second table gives the quantitative compositions of the three dosage strengths that contain 1.8 mg prednisolone.

TABLE 4
Composition of Drug Product Dosage Form
Containing 0.9 mg Prednisolone
Quantity per Capsule

| Ingredient | Function | Standard | 0.9/45 mg | 0.9/90 mg | 0.9/180 mg |
|---|---|---|---|---|---|
| Prednisolone anhydrous | Active | USP/EP | 0.90 mg | 0.90 mg | 0.90 mg |
| micronized | | | | | |
| Dipyridamole | Active | USP/EP/BP | 45.00 mg | 90.00 mg | 180.00 mg |
| Microcrystalline | Carrier for | USP/NF/EP | 87.03 mg | 87.03 mg | 87.03 mg |
| cellulose (Celphere CP- | prednisolone | | | | |
| 708) | | | | | |
| Microcrystalline | Diluent | USP/NF/EP | 11.30 mg | 22.54 mg | 45.10 mg |
| cellulose | | | | | |
| (Avicel PH 102) | | | | | |
| Polyvinylpyrrolidone | Binder | USP/EP | 3.29 mg | 5.99 mg | 11.39 mg |
| (Kollidon 30) | | | | | |
| Pregelatinized starch | Diluent, | USP | 11.30 mg | 22.54 mg | 45.10 mg |
| | binder | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

TABLE 5
Composition of Drug Product Dosage Form
Containing 1.8 mg Prednisolone
Quantity per Capsule

| Ingredient | Function | Standard | 1.8/45 mg | 1.8/90 mg | 1.8/180 mg |
|---|---|---|---|---|---|
| Prednisolone anhydrous | Active | USP/EP | 1.80 mg | 1.80 mg | 1.80 mg |
| micronized | | | | | |
| Dipyridamole | Active | USP/EP/BP | 45.00 mg | 90.00 mg | 180.00 mg |
| Microcrystalline | Carrier for | USP/NF/EP | 87.03 mg | 87.03 mg | 87.03 mg |
| cellulose (Celphere CP- | prednisolone | | | | |
| 708) | | | | | |
| Microcrystalline | Diluent | USP/NF/EP | 11.30 mg | 22.54 mg | 45.10 mg |
| cellulose | | | | | |
| (Avicel PH 102) | | | | | |
| Polyvinylpyrrolidone | Binder | USP/EP | 3.87 mg | 6.57 mg | 11.97 mg |
| (Kollidon 30) | | | | | |
| Pregelatinized starch | Diluent, | USP | 11.30 mg | 22.54 mg | 45.10 mg |
| | binder | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

### Manufacturing Process

The manufacturing process for formulations of the combinations of the invention includes three manufacturing steps followed by packaging: the manufacture of prednisolone beads, the manufacture of dipyridamole beads, and the manufacture of capsules and packaging.

Prednisolone Bead Manufacturing Process

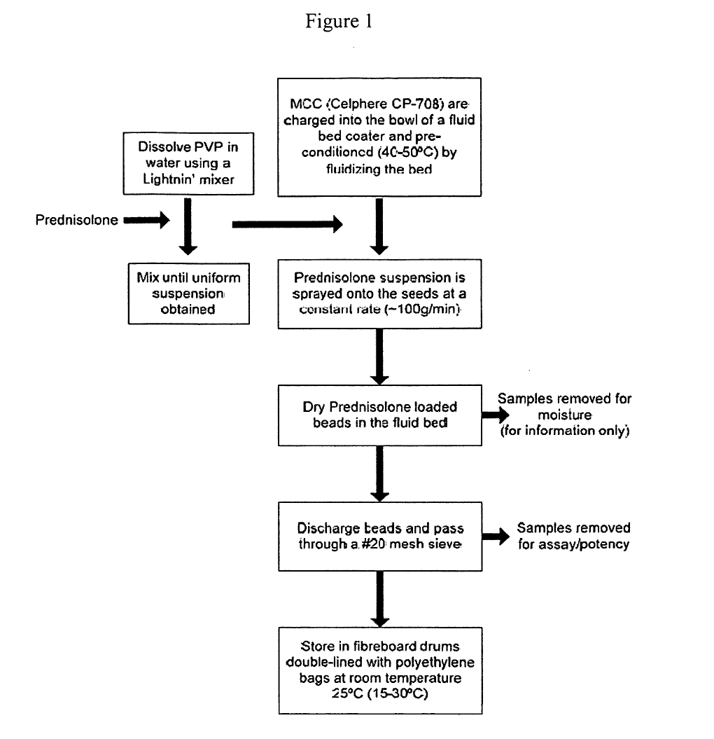

The prednisolone beads are manufactured by coating non-pareil seeds with prednisolone. The process is described in greater detail below and is shown schematically in FIG. 1. PVP (Kollidon 30) is dissolved in purified water using a Lightnin' mixer, or other similar mixer. Prednisolone is then added to the solution of PVP and water and mixed until a uniform suspension is formed. Non-pareil seeds of MCC (Celphere CP-708) are charged into the bowl of a fluid bed coater and pre-conditioned to temperature of 40-50° C. by fluidizing the bed. The prednisolone suspension is sprayed onto the fluidizing pre-conditioned non-pareil seeds at a constant rate of ˜100 g/minute ensuring that there is no agglomeration of the beads due to excessive wetting. Care is taken to ensure that an appropriate spray rate is maintained so as to prevent spray drying of prednisolone. The product bed temperature is maintained within the range of 40-50° C. by maintaining the inlet air temperature range of 60-70° C. Upon completion of the spray process, the prednisolone loaded beads are dried to a moisture content of less than 2%. The dried beads are discharged and screened through a #20 mesh sieve to remove any agglomerates. The screened beads are stored at room temperature 25° C. (15 to 30° C.) in fiber-board drums double lined with polyethylene bags. The prednisolone beads are analyzed for potency (assay) to determine the appropriate fill weight for the manufacture of the capsules. Table 6 summarizes the quantitative compositions of prednisolone capsules.

TABLE 6
Composition of Prednisolone Capsules

| Ingredient | Function | Standard | 0.9 mg | 1.8 mg |
|---|---|---|---|---|
| Prednisolone | Active | USP/EP | 0.9 mg | 1.80 mg |
| anhydrous | | | | |
| micronized | | | | |
| Microcrystalline | Carrier for | USP/ | 87.03 mg | 87.03 mg |
| cellulose (Celphere | prednisolone | NF/EP | | |
| CP-708) | | | | |
| Polyvinylpyrrolidone | Binder | USP/EP | 0.585 mg | 1.17 mg |
| (Kollidon 30) | | | | |
| Purified water^{b} | Granulating | USP | QS | QS |
| | agent | | | |

^{b}Removed during processing
Abbreviations:
EP = European Pharmacopeia;
NF = National Formulary;
QS = quantity sufficient;
USP = United States Pharmacopeia

Dipyridamole Homogenous Bead Manufacturing Process

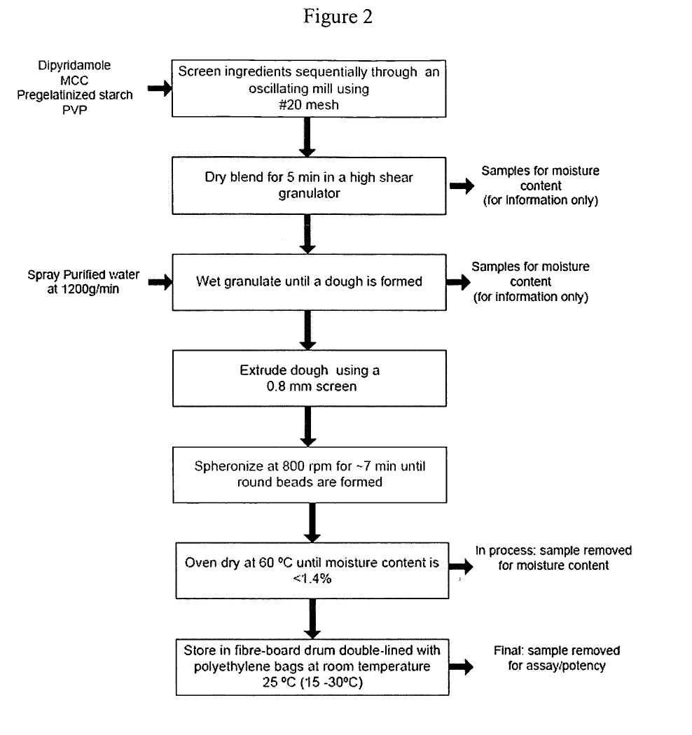

The dipyridamole beads are manufactured by extrusion spheronization. The manufacturing process for the dipyridamole beads is described in greater detail below and is shown schematically in FIG. 2. Dipyridamole is screened using an oscillating mill fitted with a #20 mesh screen and transferred into the bowl of a high shear granulator. MCC, pregelatinized starch and PVP are added to the oscillating mill successively to wash out any remaining dipyridamole. The milled materials are transferred into the bowl of a high shear granulator where they are dry blended for 5 minutes. A moisture sample of the dry blend is taken for information purposes only. The dry dipyridamole mix is then wet granulated using purified water as the granulating agent at a spray rate of 1200 g/minute till a dough is formed. Samples are removed for determination of moisture content. The wet mass of the dipyridamole dough is passed through the 0.8 mm screen of the extruder and spheronized for about 7 minutes at 800 revolutions per minute (rpm) until rounded beads are formed. The wet beads are dried in an oven set at 60° C. until the moisture content is less than 1.4%. The dried beads are stored at room temperature 25° C. (15-30° C.) in fiber-board drums double lined with polyethylene bags. The final beads are analyzed for potency (assay) to determine the appropriate fill weight for capsules. Table 7 summarizes the quantitative compositions of dipyridamole capsules.

TABLE 7
Composition of Capsules Containing Dipyridamole Homogenous
Beads
Quantity per Capsule

| Ingredient | Function | Standard | 45 mg | 90 mg | 180 mg |
|---|---|---|---|---|---|
| Dipyridamole | Active | USP/EP/BP | 45.00 mg | 90.00 mg | 180.00 mg |
| Microcrystalline cellulose | Diluent | USP/NF/EP | 11.30 mg | 22.54 mg | 45.10 mg |
| (Avicel PH 102) | | | | | |
| Pregelatinized starch | Diluent, | USP | 11.30 mg | 22.54 mg | 45.10 mg |
| | binder | | | | |
| Polyvinylpyrrolidone | Binder | USP/EP | 2.70 mg | 5.40 mg | 10.80 mg |
| (Kollidon 30) | | | | | |
| Microcrystalline cellulose | filler | USP/NF/EP | 100 mg | — | — |
| (Celphere CP-708) | | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

Dipyridamole Coated Bead Manufacturing Process

The invention features controlled release dipyridamole (DP) beads. Examples of such beads include tartaric acid beads coated with dipyridamole (for example at a ratio of dipyridamole to tartaric acid of 1:0.8). Such beads are further coated with a controlled release coating. Suitable materials for the release controlling layer include EUDRAGIT RL®, EUDRAGIT RS®, cellulose derivatives such as ethylcellulose aqueous dispersions (AQUACOAT®, SURELEASE®), hydroxyethyl cellulose, hydroxypropyl cellulose, hydroxypropyl methylcellulose, polyvinylpyrrolidone, polyvinylpyrrolidone/vinyl acetate copolymer, and OPADRY®. Examples of manufacturing processes for the production of dipyridamole-coated acid beads (e.g., tartaric acid beads) are set forth in the following examples.

### Capsule Manufacturing Process

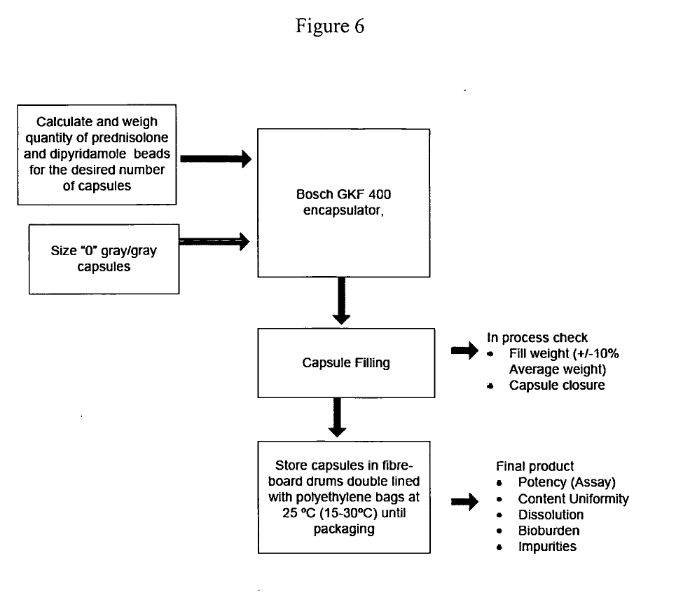

The capsule manufacturing process is described below and shown schematically in FIG. 6. The fill weight of each capsule is calculated based upon the percent weight/weight potency values of the prednisolone and dipyridamole beads. The quantity of each type of bead for the desired number of capsules is weighed and added to the Bosch GKF 400 encapsulator along with empty capsules. The prednisolone and dipyridamole beads are filled into size “0” gray/gray capsules. During the encapsulation process, capsules are checked at pre-determined intervals for fill weight variation and proper capsule closure. The machine is adjusted if any deviation is found in the established fill weight. The filled capsules are stored at room temperature conditions of 25° C. (15 to 30° C.) in fiber-board drums double lined with polyethylene bags. The final capsules are tested for identity of the active ingredients, potency of prednisolone and dipyridamole, content uniformity, dissolution, presence and quantities of related substances and bioburden prior to release.

### Packaging

Dipyridamole/prednisolone capsules are packaged in blister packs using an Uhlman packaging machine. Bulk capsules are placed on a tray of the Uhlman packager to flood feed the blister cavities. The sealing layers are placed over strips containing five capsules each and are heat sealed into place. The sealed strips are inspected at the beginning and end of the process and at 30 minute intervals during the process for proper seals and missed cavities and placed into a labeled holding container if found satisfactory. The holding container is stored in the warehouse for secondary packaging.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### Variant B

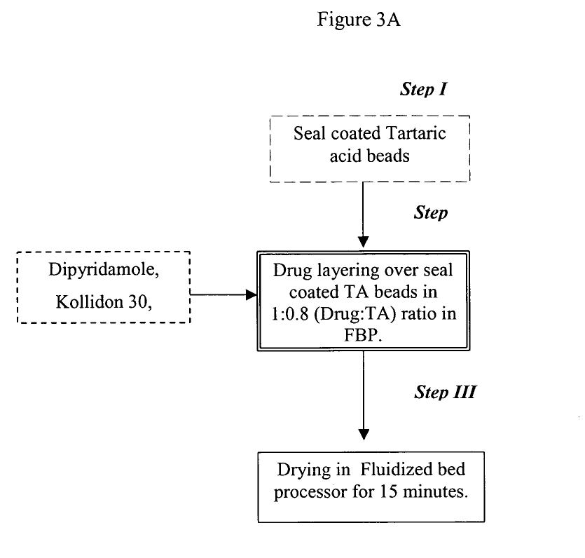

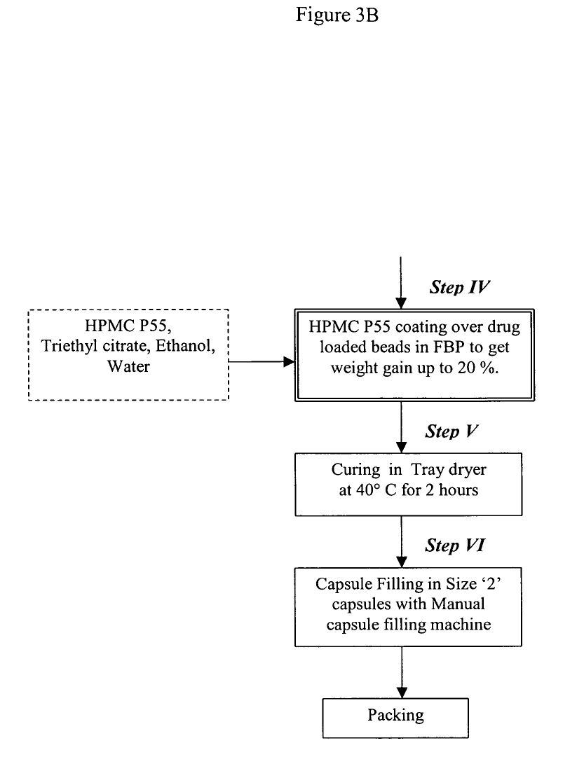

Components used in the manufacture of dipyridamole beads with a controlled release coating of hydroxypropyl methylcellulose phthalate 55 are set forth in Tables 8-11 (Variant B). The manufacturing process is depicted schematically in FIG. 3 and described in more detail below.

Manufacturing begins with the fluid bed coating of Cellets, or alternative hthalate seeds, using a coating solution consisting of tartaric acid, Pharmacoat 603, isopropyl alcohol and water. The layering process continues until there is a total of 89.1% w/w of tartaric acid loaded onto the cores. The 89.1% tartaric acid pellets are then coated in the fluid bed with a protective seal coat consisting of Kollidon 30, talc, isopropyl alcohol and water to a level of 20% weight gain.

Drug Loading

A dispersion consisting of dipyridamole, Kollidon 30 and water is sprayed onto the seal coated tartaric acid cores using the fluid bed. The amount sprayed onto these cores allows for a final ratio of 1:0.8 (dipyridamole:tartaric acid).

Modified Release Coating

A coating solution consisting of hydroxypropyl methylcellulose hthalate 55 (HPMC P-55), triethyl citrate, ethanol and water is sprayed onto the dipyridamole layered pellets. The theoretical weight gain of the modified release coating sprayed onto the DP pellets is 20%. The coated pellets are then cured in a tray drying oven for 2 hours at 40° C.

Protocol

The following is an exemplary protocol for manufacturing the hydroxypropyl methylcellulose hthalate 55 coated beads.

Preparation of Drug Suspension

Dissolve Kollidon 30 in isopropyl alcohol using a overhead stirrer under vortex to get a clear solution. Disperse dipyridamole (passed through #40 sieve) in the above solution to obtain a homogenous suspension. Strain suspension through #60 sieve. Arrange the Fluid Bed Processor with the bottom spray and the wurster column. Load the wurster with seal coated tartaric acid beads. Spray the suspension of dipyridamole onto the tartaric acid beads using a peristaltic pump at a desired spray rate. Ensure that the suspension remains stirring throughout the coating process. Coat the tartaric acid bead with the drug suspension. After spraying is complete, dry the drug layered beads in the fluid bed.

Preparation of Delayed Release Coating Suspension

Dissolve HPMC P-55 in a mixture of ethanol and purified water using an over-head stirrer under vortex stirring. Add triethyl citrate and stir the solution for 20 minutes. Pass the solution through #80 sieve and use for coating.

Delayed Release Coating

Arrange the Fluid Bed Processor with the bottom spray and the wurster column. Load the wurster with drug loaded beads. Spray the polymer solution onto the drug loaded beads using a peristaltic pump at a desired spray rate ensure that the coating solution is stirred throughout the coating process. Dry and cure the polymer coated beads for 2 hours.

TABLE 8
Excipients

| Excipient | Chemical Name | Function |
|---|---|---|
| Dipyridamole | — | API |
| Kollidon 30 | Polyvinylpyrrolidone | Non Functional |
| | | Coating Agent |
| Cellets | Microcrystalline | Non-Pareil |
| | Cellulose Spheres | Seed |
| Tartaric Acid | — | Solubilizing |
| | | Agent |
| Pharmacoat 603 | Hypromellose | Binder |
| Hydroxypropylmethylcellulose | — | Functional |
| Pthalate 55 | | Coating |
| | | Agent |
| Triethyl Citrate | — | Plasticizer |
| Talc | — | Glidant |
| Isopropyl Alcohol* | — | Solvent |
| Purified Water* | — | Solvent |
| Ethanol* | — | Solvent |

*Included in the process, but not the final product

TABLE 9
Quantity of Compounds per Capsule

| | | Qty per capsule |
|---|---|---|
| | | (mg) |
| I. | Drug Loading | |
| 1. | Dipyridamole USP | 100.00 |
| 2. | Polyvinylpyrrolidone K30 USP | 28.60 |
| | Kollidon 30-BASF | |
| 3. | Seal coated Tartaric acid beads | 107.90 |
| | (74.2% Tartaric acid) | |
| II | Delayed Release Coating | |
| 4. | Hydroxy propyl methyl cellulose - Pthalate 55 | 39.50 |
| 5. | Triethyl citrate USP | 4.00 |
| | Total | 280.00 |

TABLE 10
Bead Components by Percent Weight

| | | Grade/ | | | |
|---|---|---|---|---|---|
| | | RMS | | Std | |
| Sr. No. | Ingredients | No. | % w/w | Batch qty | Units |
| 1 | ⁺Dipyridamole | USP | 35.0 | | g |
| 2. | Kollidon-30 | USP | 10.0 | 74.3 | g |
| 3. | Isopropyl | IP | 55.0 | 408.6 | g |
| | alcohol (IPA) | | | | |
| | Total | | 100.0 | | g |
| 4. | * Seal coated | | | 215.8 | g |
| | Tartaric acid | | | | |
| | beads (74.2% | | | | |
| | Tartaric acid) | | | | |

TABLE 11
Delayed Release Coating by Percent Weight

| | | Grade/ | | | |
|---|---|---|---|---|---|
| | | RMS | | Std | |
| Sr. No. | Ingredients | No. | % w/w | Batch qty | Units |
| 5. | Hydroxy | USP | 5.0 | 180.6 | g |
| | propyl methyl | | | | |
| | cellulose | | | | |
| | Pthalate 55 | | | | |
| 6. | Triethyl citrate | USP | 0.5 | 18.06 | g |
| 7. | Ethanol | IP | 75.5 | 2727.06 | g |
| 8. | Purified water | IP | 19.0 | 686.28 | g |
| | Total | | 100.0 | 3612 | g |
| 9. | Hard Gelatin | | — | 64.5 g eqv | g |
| | Capsules size | | | to 4000 | |
| | “2” | | | capsule | |

| | | | |
|---|---|---|---|
| 10. | Silica gel bags (50 g) | 07 | nos |

### EXAMPLE 2

### Variant C

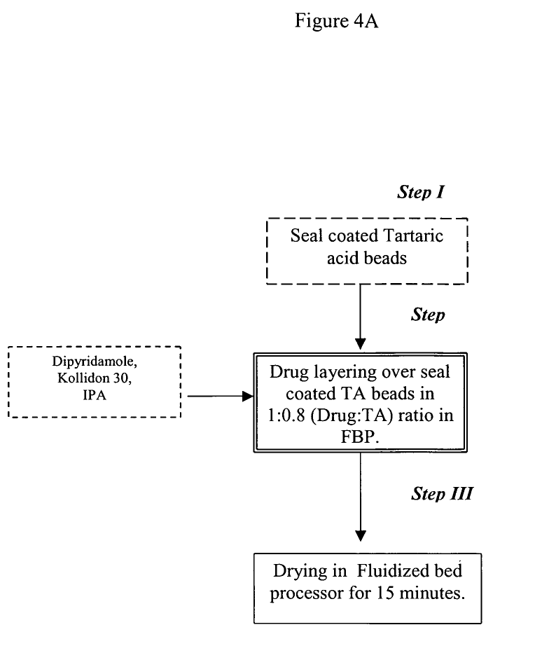

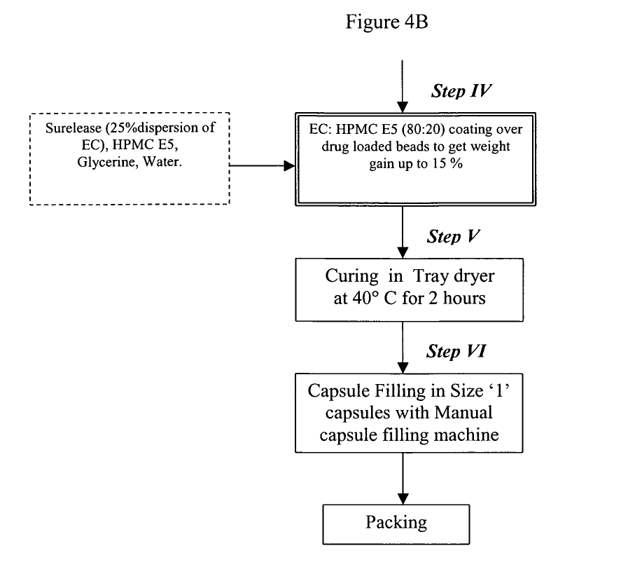

Components used in the manufacture of dipyridamole beads with a controlled release coating of of Surelease® and HPMC E5 (in a ratio of 80:20) are set forth in tables 12-14 (Variant C). The manufacturing process is depicted schematically in FIG. 4 and described in more detail below.

Manufacturing begins with the fluid bed coating of Cellets using a coating solution consisting of tartaric acid, Pharmacoat 603, isopropyl alcohol and water. The layering process continues until there is a total of 89.1% w/w of tartaric acid loaded onto the cores. The 89.1% tartaric acid pellets are then coated in the fluid bed with a protective seal coat consisting of Kollidon 30, Talc, isopropyl alcohol and water to a level of 20% weight gain.

Drug Loading

A dispersion consisting of dipyridamole, Kollidon 30 and water is sprayed onto the seal coated tartaric acid cores using the fluid bed. The amount sprayed onto these cores allows for a final ratio of 1:0.8 (dipyridamole:tartaric Acid).

Modified Release Coating

A coating solution consisting of Surelease®:HPMC E5 (80:20), glycerine and water is sprayed onto the dipyridamole layered pellets. The theoretical weight gain of the modified release coating sprayed onto the DP pellets is 15%. The coated pellets are then dried and cured for 2 hours.

Protocol

Dissolve Kollidon 30 in isopropyl alcohol using a overhead stirrer under vortex to get a clear solution. Disperse dipyridamole (passed through #40 sieve) in the above solution to obtain a homogenous suspension. Strain suspension through #60 sieve. Arrange the Fluid Bed Processor with the bottom spray and the wurster column. Load the wurster with seal coated tartaric acid beads. Spray the suspension of dipyridamole onto the tartaric acid beads using a peristaltic pump at a desired spray rate. Ensure that the suspension remains stirring throughout the coating process. After spraying is complete, dry the drug layered beads in the fluid bed.

Preparation of Modified Release Coating Suspension

Dissolve HPMC E5 in water at 60-70° C. using an overhead stirrer. Cool solution until it attains room temperature and add glycerine while stirring. Dilute the solution to the required concentration on Surelease by adding water. Pass solution through #80 sieve and use for coating. Continue to stir the solution throughout the coating process. Dry and cure the coated beads for 2 hours.

TABLE 12
Excipients

| Excipient | Chemical Name | Function |
|---|---|---|
| Dipyridamole | — | API |
| Kollidon 30 | Polyvinylpyrrolidone | Non Functional Coating |
| | | Agent |
| Cellets | Microcrystalline | Non-Pareil Seed |
| | Cellulose Spheres | |
| Tartaric Acid | — | Solubilizing Agent |
| Pharmacoat 603 | Hypromellose | Binder |
| HPMC E5 | Hypromellose (5 cps) | Functional Coating |
| | | Agent |
| Surelease ® | Ethyl cellulose | Functional Coating |
| | dispersion | Agent |
| Glycerin | — | Plasticizer |
| Talc | — | Glidant |
| Isopropyl Alcohol* | — | Solvent |
| Purified Water* | — | Solvent |

*Included in the process, but not the final product

TABLE 13
Quantity of Compounds per Capsule

| | | Qty per capsule |
|---|---|---|
| | | (mg) |
| I. | Drug Loading | |
| 1. | Dipyridamole USP | 100.00 |
| 2. | Polyvinylpyrrolidone K30 USP | 28.60 |
| | Kollidon 30-BASF | |
| 3. | Seal coated Tartaric acid beads | 107.90 |
| | (74.2% Tartaric acid) | |
| II | Modified Release Coating | |
| 4. | Ethyl cellulose | 27.97 |
| 5. | Hydroxypropylmethylcellulose 5 cps | 7.0 |
| 6. | Glycerine | 0.53 |
| | Total | 272.0 |

TABLE 14
Delayed Release Coating by Percent Weight

| | | Grade/ | % | Std Batch | |
|---|---|---|---|---|---|
| Sr. No | Ingredients | RMS No. | w/w | qty | Units |
| 5. | Surelease ® | In house | 33.56 | 469.51 | g |
| | (25% aqs | | | | |
| | dispersion of | | | | |
| | ethyl | | | | |
| | cellulose | | | | |
| 6. | HPMC E5 | USP | 2.10 | 29.38 | g |
| 7. | Glycerine | USP | 0.16 | 2.24 | g |
| 8. | Water | IP | 64.18 | 897.88 | g |
| | Total | | 100.0 | 1399.01 | g |
| 9. | Hard Gelatin | In house | — | 304 g | g |
| | Capsules | | | eqv to 4000 | |
| | size “1” | | | capsule | |

| | | | |
|---|---|---|---|
| 10. | Silica gel bags (50 g) | 07 | nos |

### EXAMPLE 3

### Variant D

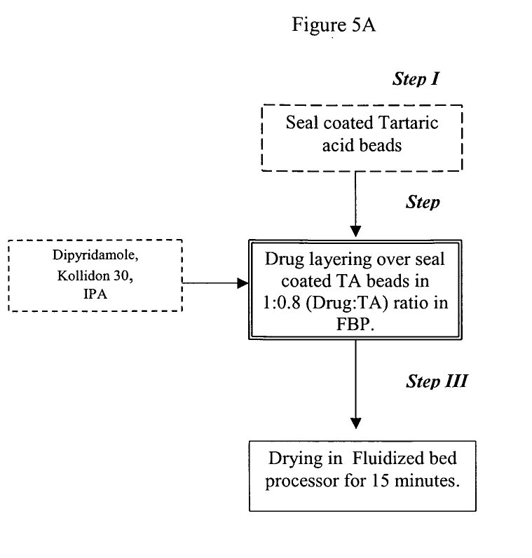

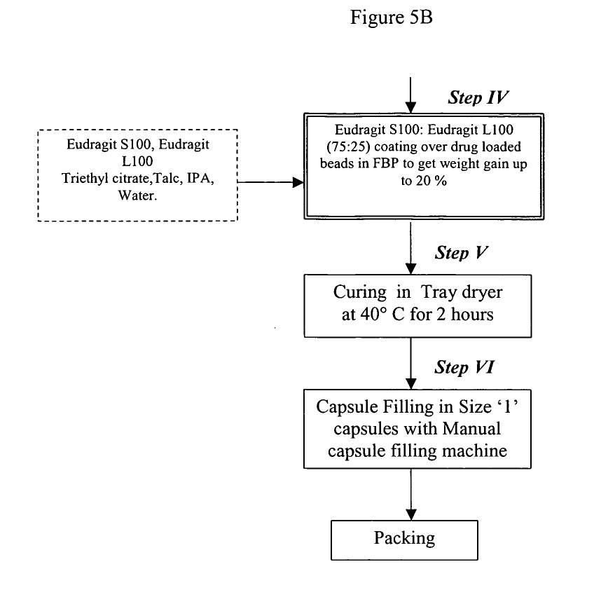

Components used in the manufacture of dipyridamole beads with a controlled release coating of Eudragit® S100 and Eudragit® L100 (in a ratio of 75:25) are set forth in Tables 15-17 (Variant D). The manufacturing process is depicted schematically in FIG. 5 and described in more detail below.

Manufacturing begins with the fluid bed coating of Cellets using a coating solution consisting of tartaric acid, Pharmacoat 603, isopropyl alcohol and water. The layering process continues until there is a total of 89.1% w/w of tartaric acid loaded onto the cores. The 89.1% tartaric acid pellets are then coated in the fluid bed with a protective seal coat consisting of Kollidon 30, talc, isopropyl alcohol and water to a level of 20% weight gain.

Drug Loading

A dispersion consisting of dipyridamole, Kollidon 30 and water is sprayed onto the seal coated tartaric acid cores using the fluid bed. The amount sprayed onto these cores allows for a final ratio of 1:0.8 (dipyridamole:tartaric acid).

Modified Release Coating

A coating solution consisting of Eudragit® S100:Eudragit® L100 (75:25), triethyl citrate, talc, isopropyl alcohol and water onto the dipyridamole layered pellets. The theoretical weight gain of the modified release coating sprayed onto the DP pellets is 20%. The coated pellets are then cured in a tray drying oven for 2 hours at 40° C.

Protocol

The following is an exemplary protocol for manufacturing the Eudragit® S100:Eudragit® L100 (75:25) coated beads.

Preparation of Drug Suspension

Dissolve Kollidon 30 in isopropyl alcohol using an overhead stirrer under vortex to get a clear solution. Disperse dipyridamole (passed through #40 sieve) in the above solution to obtain a homogenous suspension. Strain suspension through #60 sieve. Arrange the Fluid Bed Processor with the bottom spray and the wurster column. Load the wurster with seal coated tartaric acid beads. Spray the suspension of dipyridamole onto the tartaric acid beads using a peristaltic pump at a desired spray rate. Ensure that the suspension remains stirring throughout the coating process. After spraying is complete, dry the beads in the fluid bed.

Preparation of Modified Release Coating Suspension

Disperse Eudragit® L100 and Eudragit® S100 in IPA using an over-head stirrer. Add purified water to suspension and stir to get clear solution. Add triethyl citrate and talc to the above solution while stirring. Pass the through #80 sieve and use for coating. Continue to stir the solution throughout the coating process. Dry and cure the coated beads for 2 hours.

TABLE 15
Excipients

| Excipient | Chemical Name | Function |
|---|---|---|
| Dipyridamole | — | API |
| Kollidon 30 | Polyvinylpyrrolidone | Non Functional Coating |
| | | Agent |
| Cellets | Microcrystalline | Non-Pareil Seed |
| | Cellulose Spheres | |
| Tartaric Acid | — | Solubilizing Agent |
| Pharmacoat 603 | Hypromellose | Binder |
| Eudragit ® L100 | Methacrylic Acid | Functional Coating |
| | Polymer | Agent |
| Eudragit ® S100 | Methacrylic Acid | Functional Coating |
| | Polymer | Agent |
| Triethyl Citrate | — | Plasticizer |
| Talc | — | Glidant |
| Isopropyl Alcohol* | — | Solvent |
| Purified Water* | — | Solvent |

*Included in the process, but not the final product

TABLE 16
Quantity of Compounds per Capsule

| | | Qty per capsule |
|---|---|---|
| | | (mg) |
| I. | Drug Loading | |
| 1. | Dipyridamole USP | 100.00 |
| 2. | Polyvinylpyrrolidone K30 USP | 28.60 |
| | Kollidon 30-BASF | |
| 3. | Seal coated Tartaric acid beads | 107.90 |
| | (74.2% Tartaric acid) | |
| II | Modified Release Coating | |
| 4. | Eudragit ® S 100 | 22.20 |
| 5. | Eudragit ® L100 | 7.40 |
| 6. | Triethyl citrate | 2.90 |
| 7. | Talc | 14.80 |
| | Total | 283.80 |

TABLE 17
Delayed Release Coating by Percent Weight

| | | Grade/ | | | |
|---|---|---|---|---|---|
| | | RMS | | Std Batch | |
| Sr. No | Ingredients | No. | % w/w | qty | Units |
| 5. | Eudragit ® | USP | 4.5 | 93.12 | g |
| | S100 | | | | |
| 6. | Eudragit ® | USP | 1.5 | 31.04 | g |
| | L100 | | | | |
| 7. | Triethyl citrate | USP | 0.60 | 12.42 | g |
| 8. | Talc | USP | 3.00 | 62.08 | g |
| 9. | Purified water | IP | 5.00 | 103.47 | g |
| 10. | Isopropyl | USP | 85.40 | 1767.18 | g |
| | alcohol | | | | |
| 11. | Total | | 100.00 | 2069.31 | g |
| 12. | Hard Gelatin | RMS/ | — | 304 g eqv | g |
| | Capsules | 169 | | to 4000 | |
| | size‘1’ | | | capsule | |

| | | | |
|---|---|---|---|
| 13 | Silica gel bags (50 g) | 07 | nos |

### EXAMPLE 4

### Variant D1

Components used in the manufacture of dipyridamole beads with a controlled release coating of Eudragit® S100 and Eudragit® L100 (in a ratio of 75:25) are set forth in Tables 18-20 (Variant D1). The manufacturing process is described in more detail below.

Manufacturing begins with the fluid bed coating of Cellets using a coating solution consisting of tartaric acid, Pharmacoat 603, isopropyl alcohol and water. The layering process continues until there is a total of 89.1% w/w of tartaric acid loaded onto the cores. The 89.1% tartaric acid pellets are then coated in the fluid bed with a protective seal coat consisting of Kollidon 30, talc, isopropyl alcohol and water to a level of 20% weight gain.

Drug Loading

A dispersion consisting of dipyridamole, Kollidon 30 and isopropyl alcohol is sprayed onto the seal coated tartaric acid cores using the fluid bed. The amount sprayed onto these cores allows for a final ratio of 1:0.8 (dipyridamole:tartaric acid).

Modified Release Coating

A coating solution consisting of Eudragit® S100:Eudragit® L100 (75:25), triethyl citrate, talc, isopropyl alcohol and water onto the dipyridamole layered pellets. The theoretical weight gain of the modified release coating sprayed onto the DP pellets is 10%. The coated pellets are then cured in a tray drying oven for 2 hours at 40° C.

Protocol

The following is an exemplary protocol for manufacturing the Eudragit® S100:Eudragit® L100 (75:25) coated beads.

Preparation of Drug Suspension

Dissolve Kollidon 30 in isopropyl alcohol using an overhead stirrer under vortex to get a clear solution. Disperse dipyridamole in the above solution to obtain a homogenous suspension. Strain suspension through #100 mesh sieve. Arrange the Fluid Bed Processor with the bottom spray and the wurster column. Load the wurster with seal coated tartaric acid beads. Spray the suspension of dipyridamole onto the tartaric acid beads using a peristaltic pump at a desired spray rate. Ensure that the suspension remains stirring throughout the coating process. After spraying is complete, dry the beads in the fluid bed.

Preparation of Modified Release Coating Suspension

Disperse Eudragit® S100 and Eudragit® L100 in water and 90% of the IPA using an over-head stirrer. Add purified water to suspension and stir to get clear solution. Add triethyl citrate and mix for at least 15 minutes. In a separate container add water, 10% of the IPA and talc, then homogenize for 10 minutes to form a dispersion. Combine the Talc dispersion and Eudragit solution and mix for at least 30 minutes prior to coating. Continue to stir the coating solution throughout the coating process. Dry and cure the coated beads for 2 hours.

TABLE 18
Excipients

| Excipient | Chemical Name | Function |
|---|---|---|
| Dipyridamole | — | API |
| Kollidon 30 | Polyvinylpyrrolidone | Non-Functional Coating |
| | | Agent/Binder |
| Cellets | Microcrystalline | Non-Pareil Seed |
| | Cellulose Spheres | |
| Tartaric Acid | — | Solubilizing Agent |
| Pharmacoat 603 | Hypromellose | Binder |
| Eudragit ® L100 | Methacrylic Acid | Functional Coating Agent |
| | Polymer | |
| Eudragit ® S100 | Methacrylic Acid | Functional Coating Agent |
| | Polymer | |
| Triethyl Citrate | — | Plasticizer |
| Talc | — | Glidant |
| Isopropyl | — | Solvent |
| Alcohol* | | |
| Purified Water* | — | Solvent |

*Included in the process, but not the final product

TABLE 19
Quantity of Compounds per Capsule

| | | Qty per capsule |
|---|---|---|
| | | (mg) |
| I. | Drug Loading | |
| 1. | Dipyridamole USP | 180.00 |
| 2. | Polyvinylpyrrolidone K30 USP | 58.21 |
| | Kollidon 30-BASF | |
| 3. | Seal coated Tartaric acid beads | 168.51 |
| | (74.2% Tartaric acid) | |
| II | Modified Release Coating | |
| 4. | Eudragit ® S 100 | 19.25 |
| 5. | Eudragit ® L100 | 6.27 |
| 6. | Triethyl citrate | 2.69 |
| 7. | Talc | 12.83 |
| | Total | 447.76 |

TABLE 20
Delayed Release Coating by Percent Weight

| Sr. | | Grade/ | | | |
|---|---|---|---|---|---|
| No. | Ingredients | RMS No. | % w/w | Std Batch qty | Units |
| 5. | Eudragit ® S100 | USP | 4.5 | 56.2 | g |
| 6. | Eudragit ® L100 | USP | 1.5 | 18.7 | g |
| 7. | Triethyl citrate | USP | 0.6 | 7.5 | g |
| 8. | Talc | USP | 3.0 | 37.4 | g |
| 9. | Purified water | IP | 5.0 | 62.6 | g |
| 10. | Isopropyl alcohol | USP | 85.4 | 1067.6 | g |
| 11. | Total | | 100.0 | 1250.0 | g |
| 12. | Hard Gelatin | RMS/ | — | 895.5 g eqv to | g |
| | Capsules size ‘0CS’ | 169 | | 2000 capsule | |

### EXAMPLE 5

### Variant D2

Components used in the manufacture of dipyridamole beads with a controlled release coating of Eudragit® S100 and Eudragit® L100 (in a ratio of 75:25) are set forth in Tables 21-23 (Variant D2). The manufacturing process is described in more detail below.

Manufacturing begins with the fluid bed coating of Cellets using a coating solution consisting of tartaric acid, Pharmacoat 603, isopropyl alcohol and water. The layering process continues until there is a total of 89.1% w/w of tartaric acid loaded onto the cores. The 89.1% tartaric acid pellets are then coated in the fluid bed with a protective seal coat consisting of HPMC Phthalate PH-55, triethyl citrate, isopropyl alcohol and acetone to a level of 15% weight gain.

Drug Loading

A dispersion consisting of dipyridamole, Kollidon 30 and isopropyl alcohol is sprayed onto the seal coated tartaric acid cores using the fluid bed. The amount sprayed onto these cores allows for a final ratio of 1:0.8 (dipyridamole:tartaric acid).

Modified Release Coating

A coating solution consisting of Eudragit® S100:Eudragit® L100 (75:25), triethyl citrate, talc, isopropyl alcohol and water onto the dipyridamole layered pellets. The theoretical weight gain of the modified release coating sprayed onto the DP pellets is 10%. The coated pellets are then cured in a tray drying oven for 2 hours at 40° C.

Protocol

The following is an exemplary protocol for manufacturing the Eudragit® S100:Eudragit® L100 (75:25) coated beads.

Preparation of Drug Suspension

Dissolve Kollidon 30 in isopropyl alcohol using an overhead stirrer under vortex to get a clear solution. Disperse dipyridamole in the above solution to obtain a homogenous suspension. Strain suspension through #100 mesh sieve. Arrange the Fluid Bed Processor with the bottom spray and the wurster column. Load the wurster with seal coated tartaric acid beads. Spray the suspension of dipyridamole onto the tartaric acid beads using a peristaltic pump at a desired spray rate. Ensure that the suspension remains stirring throughout the coating process. Drying: After spraying is complete, dry the beads in the fluid bed.

Preparation of Modified Release Coating Suspension

Disperse Eudragit® S100 and Eudragit® L100 in water and 90% of the IPA using an over-head stirrer. Add purified water to suspension and stir to get clear solution. Add triethyl citrate and mix for at least 15 minutes. In a separate container add water, 10% of the IPA and talc, then homogenize for 10 minutes to form a dispersion. Combine the Talc dispersion and Eudragit solution and mix for at least 30 minutes prior to coating. Continue to stir the coating solution throughout the coating process. Dry and cure the coated beads for 2 hours.

TABLE 21
Excipients

| Excipient | Chemical Name | Function |
|---|---|---|
| Dipyridamole | — | API |
| Kollidon 30 | Polyvinylpyrrolidone | Binder |
| Cellets | Microcrystalline | Non-Pareil Seed |
| | Cellulose Spheres | |
| Tartaric Acid | — | Solubilizing Agent |
| HPMC Phthalate PH-55 | Hypromellose Phthalate | Non-Functional |
| | | Coating Agent |
| Pharmacoat 603 | Hypromellose | Binder |
| Eudragit ® L100 | Methacrylic Acid | Functional Coating |
| | Polymer | Agent |
| Eudragit ® S100 | Methacrylic Acid | Functional Coating |
| | Polymer | Agent |
| Triethyl Citrate | — | Plasticizer |
| Talc | — | Glidant |
| Isopropyl Alcohol* | — | Solvent |
| Acetone* | — | Solvent |
| Purified Water* | — | Solvent |

*Included in the process, but not the final product

TABLE 22
Quantity of Compounds per Capsule

| | | Qty per capsule |
|---|---|---|
| | | (mg) |
| I. | Drug Loading | |
| 1. | Dipyridamole USP | 180.00 |
| 2. | Polyvinylpyrrolidone K30 USP | 33.37 |
| | Kollidon 30-BASF | |
| 3. | Seal coated Tartaric acid beads | 185.80 |
| | (77.5% Tartaric acid) | |
| II | Modified Release Coating | |
| 4. | Eudragit ® S 100 | 18.88 |
| 5. | Eudragit ® L100 | 6.15 |
| 6. | Triethyl citrate | 2.53 |
| 7. | Talc | 12.29 |
| | Total | 439.02 |

TABLE 23
Delayed Release Coating by Percent Weight

| | | Grade/ | | | |
|---|---|---|---|---|---|
| Sr. | | RMS | | | |
| No. | Ingredients | No. | % w/w | Std Batch qty | Units |
| 5. | Eudragit ® S100 | USP | 4.5 | 56.2 | g |
| 6. | Eudragit ® L100 | USP | 1.5 | 18.7 | g |
| 7. | Triethyl citrate | USP | 0.6 | 7.5 | g |
| 8. | Talc | USP | 3.0 | 37.4 | g |
| 9. | Purified water | IP | 5.0 | 62.6 | g |
| 10. | Isopropyl alcohol | USP | 85.4 | 1067.6 | g |
| 11. | Total | | 100.0 | 1250.0 | g |
| 12. | Hard Gelatin | RMS/ | — | 878.0 g eqv to | g |
| | Capsules size‘0CS’ | 169 | | 2000 capsule | |

### EXAMPLE 6

### Variant E (Cofilled Capsules)

Components used in the manufacture of prednisolone beads with a controlled release coating of Eudragit® S100 and Eudragit® L100 (in a ratio of 75:25) are set forth in Tables 24-26 (Variant E). The manufacturing process is described in more detail below.

Drug Loading

A solution consisting of prednisolone, Kollidon 30 and water is sprayed onto Cellets using the fluid bed. The amount sprayed onto these cores allows for a final prednisolone amount to be 2.0%. Some 2.0% prednisolone pellets are set aside to be used as the IR portion and some will be used for further processing to manufacture the delayed release portion.

Seal Coating

A solution consisting of Kollidon VA-64, Pharmacoat 603 and water is sprayed onto prenisolone coated pellets using the fluid bed. The amount sprayed onto these cores allows for a final prednisolone amount to be 1.9%. The 1.9% prednisolone pellets are then further coated with a delayed release coating.

Delayed Release Coating

A coating solution consisting of Eudragit® S100:Eudragit® L100 (75:25), triethyl citrate, talc, isopropyl alcohol and water onto seal coated prednisolone layered pellets. The theoretical weight gain of the modified release coating sprayed onto the DP pellets is 25%. The coated pellets are then cured in a tray drying oven for 8 hours at 40° C.

Protocol

The following is an exemplary protocol for manufacturing the Eudragit® S100:Eudragit® L100 (75:25) coated beads.

Preparation of Drug Suspension

Dissolve Kollidon 30 in water using an overhead stirrer under vortex to get a clear solution. Disperse prednisolone in the above solution to obtain a solution. Arrange the Fluid Bed Processor with the bottom spray and the wurster column. Load the wurster with Cellets. Spray the prednisolone solution onto the Cellets using a peristaltic pump at a desired spray rate. Ensure that the solution remains stirring throughout the coating process. Drying: After spraying is complete, dry the beads in the fluid bed.

Preparation of Seal Coating Solution

Dissolve Kollidon VA-64 in water and isopropyl alcohol using an overhead stirrer under vortex to get a clear solution. Disperse Pharmacoat 603 in the above solution and mix until dissolved. Arrange the Fluid Bed Processor with the bottom spray and the wurster column. Load the wurster with prednisolone coated pellets. Spray the seal coat solution onto the prednisolone pellets using a peristaltic pump at a desired spray rate. Ensure that the solution remains stirring throughout the coating process. Drying: After spraying is complete, dry the beads in the fluid bed.

Preparation of Modified Release Coating Suspension

Disperse Eudragit® S100 and Eudragit® L100 in water and 90% of the IPA using an over-head stirrer. Add purified water to suspension and stir to get clear solution. Add triethyl citrate and mix for at least 15 minutes. In a separate container add water, 10% of the IPA and talc, then homogenize for 10 minutes to form a dispersion. Combine the Talc dispersion and Eudragit solution and mix for at least 30 minutes prior to coating. Continue to stir the coating solution throughout the coating process. Dry and cure the coated beads for 8 hours

TABLE 24
Excipients

| Excipient | Chemical Name | Function |
|---|---|---|
| Prednisolone | — | API |
| Kollidon 30 | Polyvinylpyrrolidone | Binder |
| Cellets | Microcrystalline | Non-Pareil Seed |
| | Cellulose Spheres | |
| Kollidon VA-64 | Vinylpyrrolidone | Non-Functional Coating |
| | | Agent |
| Pharmacoat 603 | Hypromellose | Binder |
| Eudragit ® L100 | Methacrylic Acid | Functional Coating |
| | Polymer | Agent |
| Eudragit ® S100 | Methacrylic Acid | Functional Coating |
| | Polymer | Agent |
| Triethyl Citrate | — | Plasticizer |
| Talc | — | Glidant |
| Isopropyl Alcohol* | — | Solvent |
| Purified Water* | — | Solvent |

*Included in the process, but not the final product

TABLE 25
Quantity of Compounds per Capsule

| | | Qty per capsule | |
|---|---|---|---|
| | | (mg) | |
| I. | IR Portion | | |
| 1. | Prednisolone USP | 1.80 | |
| 2. | Polyvinylpyrrolidone K30 USP | 1.17 | |
| | Kollidon 30-BASF | | |
| 3. | Cellets | 87.03 | |
| | Total | 90.00 | mg |
| II | DR Portion | | |
| 4. | Prednisolone USP | 0.90 | |
| 5. | Polyvinylpyrrolidone K30 USP | 0.59 | |
| | Kollidon 30-BASF | | |
| 6. | Cellets | 41.74 | |
| 7. | Vinylpyrrolidone USP | 0.56 | |
| | Kollidon VA-64-BASF | | |
| 8. | Pharmacoat 603 | 0.84 | |
| 9. | Eudragit ® S 100 | 5.46 | |
| 10 | Eudragit ® L100 | 1.80 | |
| 11. | Triethyl citrate | 0.70 | |
| 12. | Talc | 3.66 | |
| | Total | 56.25 | mg |

TABLE 26
Delayed Release Coating by Percent Weight

| | | Grade/ | | | |
|---|---|---|---|---|---|
| Sr. | | RMS | | Std Batch | |
| No. | Ingredients | No. | % w/w | qty | Units |
| 1. | Eudragit ® | USP | 4.5 | 937.8 | g |
| | S100 | | | | |
| 2. | Eudragit ® | USP | 1.5 | 312.4 | g |
| | L100 | | | | |
| 3. | Triethyl citrate | USP | 0.6 | 124.8 | g |
| 4. | Talc | USP | 3.0 | 625.7 | g |
| 5. | Purified water | IP | 5.9 | 1229.4 | g |
| 6. | Isopropyl | USP | 84.5 | 17609.4 | g |
| | alcohol | | | | |
| 7. | Total | | 100.0 | 20839.5 | g |
| 8. | Hard Gelatin | RMS/ | — | 720.0 g (IR) + | g |
| | Capsules | 169 | | 450 g (DR) | |
| | size‘3CS’ | | | eqv to 8000 | |
| | | | | capsule | |

### EXAMPLE 7

### Variant F (Combination Pellet)

Components used in the manufacture of prednisolone beads with a controlled release coating of Eudragit® S100 and Eudragit® L100 (in a ratio of 75:25) are set forth in Tables 27-29 (Variant F). The manufacturing process is described in more detail below.

Drug Loading

A solution consisting of prednisolone, Kollidon 30 and water is sprayed onto Cellets using the fluid bed. The amount sprayed onto these cores allows for a final prednisolone amount to be 2.5%. The 2.5% prednisolone pellets are then further coated in order to contain an immediate release and delayed release functions.

Seal Coating

A solution consisting of Kollidon VA-64, Pharmacoat 603 and water is sprayed onto prenisolone coated pellets using the fluid bed. The amount sprayed onto these cores allows for a final prednisolone amount to be 2.4%. The 2.4% prednisolone pellets are then further coated in order to contain an immediate release and delayed release functions.

Delayed Release Coating

A coating solution consisting of Eudragit® S100:Eudragit® L100 (75:25), triethyl citrate, talc, isopropyl alcohol and water onto seal coated prednisolone layered pellets. The theoretical weight gain of the modified release coating sprayed onto the DP pellets is 25%. The coated pellets are then cured in a tray drying oven for 8 hours at 40° C.

Second Drug Loading

A solution consisting of prednisolone, Kollidon 30 and water is sprayed onto DR coated prednisolone pellets using the fluid bed. The amount sprayed onto these cores allows for a final prednisolone amount to be 5.4% total.

Protocol

The following is an exemplary protocol for manufacturing the Eudragit® S100:Eudragit® L100 (75:25) coated beads.

Preparation of Drug Suspension

Dissolve Kollidon 30 in water using an overhead stirrer under vortex to get a clear solution. Disperse prednisolone in the above solution to obtain a solution. Arrange the Fluid Bed Processor with the bottom spray and the wurster column. Load the wurster with Cellets. Spray the prednisolone solution onto the Cellets using a peristaltic pump at a desired spray rate. Ensure that the solution remains stirring throughout the coating process. Drying: After spraying is complete, dry the beads in the fluid bed.

Preparation of Seal Coating Solution

Dissolve Kollidon VA-64 in water and isopropyl alcohol using an overhead stirrer under vortex to get a clear solution. Disperse Pharmacoat 603 in the above solution and mix until dissolved. Arrange the Fluid Bed Processor with the bottom spray and the wurster column. Load the wurster with prednisolone coated pellets. Spray the seal coat solution onto the prednisolone pellets using a peristaltic pump at a desired spray rate. Ensure that the solution remains stirring throughout the coating process. Drying: After spraying is complete, dry the beads in the fluid bed.

Preparation of Modified Release Coating Suspension

Disperse Eudragit® S100 and Eudragit® L100 in water and 90% of the IPA using an over-head stirrer. Add purified water to suspension and stir to get clear solution. Add triethyl citrate and mix for at least 15 minutes. In a separate container add water, 10% of the IPA and talc, then homogenize for 10 minutes to form a dispersion. Combine the Talc dispersion and Eudragit solution and mix for at least 30 minutes prior to coating. Continue to stir the coating solution throughout the coating process. Dry and cure the coated beads for 8 hours

TABLE 27
Excipients

| Excipient | Chemical Name | Function |
|---|---|---|
| Prednisolone | — | API |
| Kollidon 30 | Polyvinylpyrrolidone | Binder |
| Cellets | Microcrystalline Cellulose | Non-Pareil Seed |
| | Spheres | |
| Kollidon VA-64 | Vinylpyrrolidone | Non-Functional Coating |
| | | Agent |
| Pharmacoat 603 | Hypromellose | Binder |
| Eudragit ® L100 | Methacrylic Acid Polymer | Functional Coating |
| | | Agent |
| Eudragit ® S100 | Methacrylic Acid Polymer | Functional Coating |
| | | Agent |
| Triethyl Citrate | — | Plasticizer |
| Talc | — | Glidant |
| Isopropyl | — | Solvent |
| Alcohol* | | |
| Purified Water* | — | Solvent |

*Included in the process, but not the final product

TABLE 28
Quantity of Compounds per Capsule

| | | Qty per capsule | |
|---|---|---|---|
| | | (mg) | |
| I. | Drug Loading | | |
| 1. | Prednisolone USP | 0.90 | |
| 2. | Polyvinylpyrrolidone K30 USP | 0.60 | |
| | Kollidon 30-BASF | | |
| 3. | Cellets | 34.95 | |
| II | Seal Coating | | |
| 4. | Vinylpyrrolidone USP | 0.45 | |
| | Kollidon VA-64-BASF | | |
| 5. | Pharmacoat 603 | 0.65 | |
| III | Delayed Release Coating | | |
| 6. | Eudragit ® S 100 | 4.35 | |
| 7. | Eudragit ® L100 | 1.45 | |
| 8. | Triethyl citrate | 0.55 | |
| 9. | Talc | 2.95 | |
| IV. | Drug Loading | | |
| 10. | Prednisolone USP | 1.80 | |
| 11. | Polyvinylpyrrolidone K30 USP | 1.20 | |
| | Kollidon 30-BASF | | |
| | Total | 50.00 | mg |

TABLE 29
Delayed Release Coating by Percent Weight

| | | Grade/ | | | |
|---|---|---|---|---|---|
| Sr. | | RMS | | | |
| No. | Ingredients | No. | % w/w | Std Batch qty | Units |
| 1. | Eudragit ® | USP | 4.5 | 937.8 | g |
| | S100 | | | | |
| 2. | Eudragit ® | USP | 1.5 | 312.4 | g |
| | L100 | | | | |
| 3. | Triethyl citrate | USP | 0.6 | 124.8 | g |
| 4. | Talc | USP | 3.0 | 625.7 | g |
| 5. | Purified water | IP | 5.9 | 1229.4 | g |
| 6. | Isopropyl | USP | 84.5 | 17609.4 | g |
| | alcohol | | | | |
| 7. | Total | | 100.0 | 20839.5 | g |
| 8. | Hard Gelatin | RMS/ | — | 400.0 g eqv to | g |
| | Capsules size | 169 | | 8000 capsule | |
| | ‘3CS’ | | | | |

### EXAMPLE 8

### Dissolution Profiles

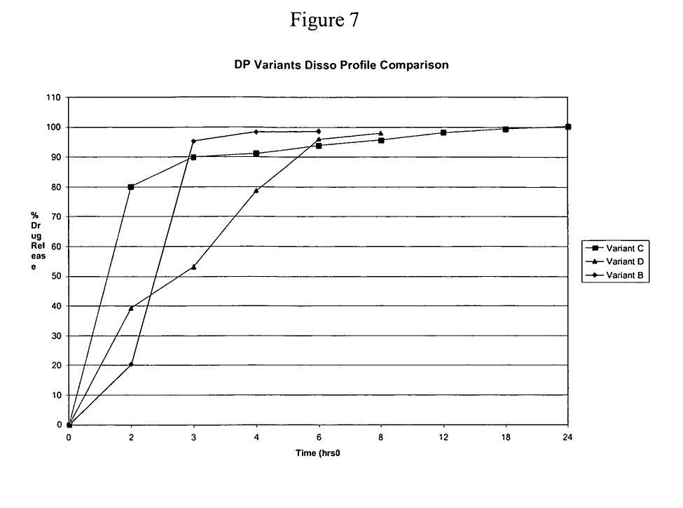

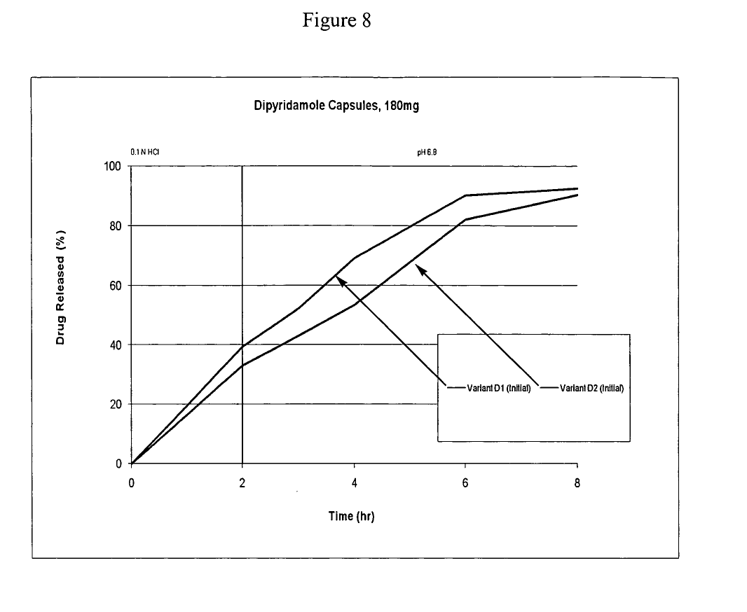

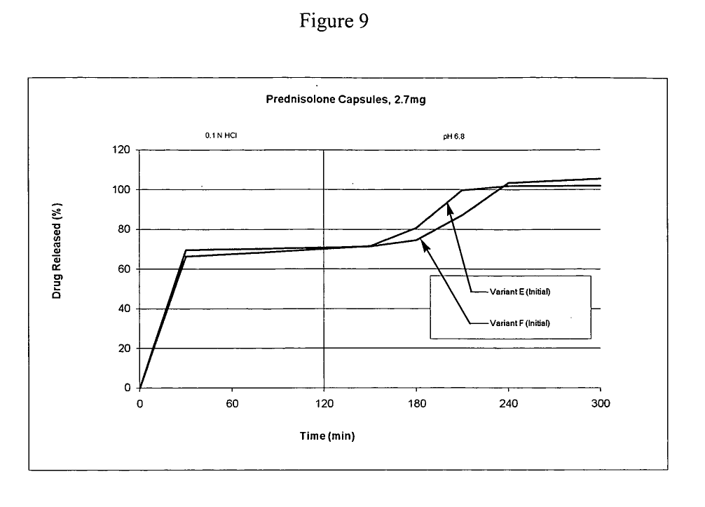

FIG. 7 is a graph depiciting the dissolution profiles for Variants B, C and D. FIG. 8 is a graph depiciting the dissolution profiles for Variants D1 and D2. FIG. 9 is a graph depiciting the dissolution profiles for Variants E and F. All of these are in measured in simulated media as described herein.

For the Variant B prototype, there is an average release of 20% dipyridamole in 0.1 N HCl within the first two hours. At the two-hour time point there is a media replacement where the prototype is added to a media containing a pH 6.8 phosphate buffer with 0.25% SLS. During this stage, the dipyridamole is release over time for a period of four hours.

For the Variant C prototype, there is an average release of 80% dipyridamole in 0.1 N HCl within the first two hours. At the two-hour time point there is a media replacement where the prototype is added to a media containing a pH 5.5 acetate buffer with 0.25% sodium lauryl sulfate. During this stage, the dipyridamole is release over time for a period of 22 hours.

For the Variant D prototype, there is an average release of 39% dipyridamole in 0.1 N HCl within the first two hours. At the two-hour time point there is a media replacement where the prototype is added to a media containing a pH 6.8 phosphate buffer with 0.25% sodium lauryl sulfate. During this stage, the dipyridamole is release over time for a period of six hours.

### EXAMPLE 9

### Headache Reduction with Reduced Absorption Rate

We have discovered that headache, a side effect of dipyridamole therapy, can be reduced by reducing the rate of rise to Cmax. To minimize the risk of headache the release of dipyridamole from the administered dosage form is modified such that in-vivo absorption rate constant (ka) is reduced (e.g., to between 0.2 to 0.90 l/hr). For the sake of comparison, the absorption rate constant (ka) for dipyridamole formulated for immediate release is in the range of 1.19 to 1.54 l/hr. Formulations that can reduce the incidence of headache include, for example, variant D. These conclusions are based upon the results of the clinical studies described below.

Clinical Trial

This trial was an open-label, balanced, randomized, four-treatment, four-sequence, four-period, single-dose crossover comparative oral bioavailability study of immediate release and modified release formulations of dipyridamole 100 mg capsules, manufactured by M/S. Rubicon Research PVT Ltd, Mumbai, India for CombinatoRx in normal, healthy, adult, human subjects after a normal breakfast.

The formulations tested in this study were:

T1: dipyridamole variant A—dipyridamole immediate-release capsules 100 mg (formula code X) (single-dose administration one 100 mg capsule in the morning per treatment period);

T2: dipyridamole variant B—modified-release capsule (single-dose administration one 100 mg capsule in the morning per treatment period);

T3: dipyridamole variant C—modified-release capsule (single-dose administration one 100 mg capsule in the morning per treatment period); and

T4: dipyridamole variant D—modified-release capsule (single-dose administration one 100 mg capsule in the morning per treatment period). Subjects were fasted overnight for at least 10 hours prior to scheduled time for a normal breakfast (about 500 cal, description provided below); dosing was done 30 minutes after the start of the breakfast. Meals or snacks were provided at 4, 8, 12, 24, 28, 32, 36 and 48 hours after dosing in each period. Seventeen blood samples were collected from each subject during each period. The venous blood samples (5 mL each) were withdrawn at pre-dose (within one and a half hours prior to normal breakfast) and at times 0.25, 0.5, 0.75, 1.0, 1.5, 2.0, 3.0, 4.0, 6.0, 8.0, 10.0, 12.0, 16.0, 24.0, 36.0 and 48.0 hours after dosing.

Plasma samples were analyzed to quantify the concentration of Dipyridamole using a validated LC/MS/MS bioanalytical method. PK Solutions 2.0™ Noncompartmental Pharmacokinetic data analysis software by Summit Research Services was used to estimate (Ka)values for both IR and Modified release DP data, which obeys two-compartment kinetics with first order absorption and elimination (best described using a triexponential curve fit).

Normal Breakfast

Dosing occurred 30 minutes after eating the normal breakfast described below.

Meal Menu
Contents of Meal ID

| | |
|---|---|
| Meal ID: 147/02 | Meal Type: Normal breakfast |

| S. No | Food Item | Portion Size (Cooked weight) |
|---|---|---|
| 1 | Toast with butter | 2 No. |
| 2 | Egg (Fried in Butter) | 1 No. |
| 3 | Milk | 1 glass |

Nutritive value of food items (Raw weight)

| S. | | Quantity | Protein | Fat | Carbohydrates |
|---|---|---|---|---|---|
| No | Food item | (gm)/(ml) | (Gram) | (Gram) | (Gram) |
| 1 | Wheat refined | 40 | 3.10 | 0.40 | 20.70 |
| 2 | Egg | 40 | 6.65 | 6.65 | 0 |
| 3 | Milk (Whole milk) | 240 | 10.32 | 15.60 | 12.00 |
| 4 | Sugar | 10 | 0 | 0 | 10 |
| 5 | Oil | 5 | 0 | 5.00 | 0 |
| 6 | Butter | 2.5 | 0 | 2.03 | 0 |

| Total | 20.07 | 29.68 | 42.70 |
|---|---|---|---|
| Energy (Kcal) | 80.28 | 267.12 | 170.80 |
| Total Energy (Kcal) | 518.20 | | |
| Percentage of Caloric Content | 15.49 | 51.55 | 32.96 |

Note:
Portion size of food item may vary depending on the amount of water added during cooking

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for treating an immunoinflammatory disorder in a subject in need thereof, said method comprising administering to said subject a unit dosage form comprising dipyridamole coated onto acid beads and formulated for controlled release.

2. The method of claim 1, wherein said dipyridamole is coated with a controlled release coating.

3. The method of claim 2, wherein said controlled release coating comprises hyrdoxypropyl methylcellulose phthalate 55, Surelease®:HPMC ES, and Eudragit® L100:Eudragit® S100.

4. The method of claim 1 wherein said unit dosage form further comprises dipyridamole formulated for immediate release.

5. The method of claim 1, wherein said unit dosage form comprises between 40 and 400 mg dipyridamole.

6. The method of claim 5, wherein said unit dosage form comprises 45 mg of dipyridamole.

7. The method of claim 5, wherein said unit dosage form comprises 90 mg of dipyridamole.

8. The method of claim 5, wherein said unit dosage form comprises 180 mg of dipyridamole.

9. The method of claim 5, wherein said unit dosage form comprises 360 mg of dipyridamole.

10. The method of claim 5, wherein 50% to 80% of said dipyridamole is formulated for controlled release and 20% to 50% of said dipyridamole is formulated for immediate release.

11. The method of claim 1, wherein said acid beads are tartaric acid beads.

12. The method of claim 11, wherein the ratio of dipyridamole to tartaric acid is 1:0.8.

13. The method of claim 1, wherein said unit dosage form is administered once or twice daily.

14. The method of claim 1, further comprising administering to said subject a corticosteroid.

15. The method of claim 14, wherein said corticosteroid is administered in two doses.

16. The method of claim 15, wherein said first dose is administered in a unit dosage formulation comprising from 1.5 to 2.5 mg of prednisolone or an equivalent, equipotent amount of another corticosteroid, and said second dose is administered in a unit dosage formulation comprising from 0.75 to 1.25 mg of prednisolone or an equivalent, equipotent amount of another corticosteroid.

17. The method of claim 16, wherein said first dose is administered in a unit dosage formulation comprising 1.8 mg of prednisolone or an equivalent, equipotent amount of another corticosteroid, and said second dose is administered in a unit dosage formulation comprising 0.9 mg of prednisolone or an equivalent, equipotent amount of another corticosteroid.

18. The method of claim 14, wherein said corticosteroid is selected from the group consisting of prednisolone, prednisone, budesonide, methylprednisolone, fluticasone, betamethasone, and deflazacort.

19. The method of claim 18, wherein said corticosteroid is prednisolone.

20. The method of claim 15, wherein said first dose is administered to said subject upon waking.

21. The method of claim 15, wherein said second dose is administered to said subject 4 to 6 hours after said first dose.

22. The method of claim 14, wherein said corticosteroid is formulated for immediate release.

23. The method of claim 14, wherein said corticosteroid is formulated for controlled release.

24. The method of claim 15, wherein said first dose is administered in a unit dosage formulation comprising from 1.0 to 2.5 mg of prednisolone or an equivalent, equipotent amount of another corticosteroid, formulated for immediate release and said second dose is administered in a unit dosage formulation comprising from 0.75 to 2.0 mg of prednisolone or an equivalent, equipotent amount of another corticosteroid, formulated for controlled release.

25. The method of claim 14, wherein said corticosteroid is formulated in a unit dosage form having a dissolution release profile under in vitro conditions in which at least 50% of the corticosteroid is released within the first 30 minutes of testing, wherein said in vitro conditions employ USP Dissolution Apparatus No. 1 at 37° C.±0.5° C. and 100 rpm in 0.1N HCl as dissolution medium for the first two hours, and a pH 6.8 phosphate buffer as the medium thereafter.

26. The method of claim 1, wherein said dipyridamole is formulated in a unit dosage form having a dissolution release profile under in vitro conditions in which at least 10-55% of the dipyridamole is released within the first two hours of testing and not less than 80% of the dipyridamole is released within 8 hours, wherein said in vitro conditions employ USP Dissolution Apparatus No. 1 at 37° C.±0.5° C. and 100 rpm in 0.1N HCl as dissolution medium for the first two hours, and a pH 6.8 phosphate buffer with 0.25% sodium lauryl sulfate as the medium thereafter.

27. The method of claim 1, wherein said dipyridamole is formulated in a unit dosage form having, upon administration to fed patients, an absorption rate constant of from 0.20 to 0.90 l/hr.

28. A pharmaceutical composition in unit dosage form comprising dipyridamole coated onto acid beads and formulated for controlled release.

29. The pharmaceutical composition of claim 28, wherein said acid beads are tartaric acid beads.

30. The pharmaceutical composition of claim 28, wherein said dipyridamole is coated with a controlled release coating.

31. The pharmaceutical composition of claim 30, wherein said controlled release coating comprises hyrdoxypropyl methylcellulose phthalate 55, Surelease®:HPMC E5, and Eudragit® L100:Eudragit® S100.

32. The pharmaceutical composition of claim 28, wherein said unit dosage form further comprises dipyridamole formulated for immediate release.

33. The pharmaceutical composition of claim 28, wherein said unit dosage form comprises between 40 and 400 mg dipyridamole.

34. The pharmaceutical composition of claim 33, wherein said unit dosage form comprises 45 mg of dipyridamole.

35. The pharmaceutical composition of claim 33, wherein said unit dosage form comprises 90 mg of dipyridamole.

36. The pharmaceutical composition of claim 33, wherein said unit dosage form comprises 180 mg of dipyridamole.

37. The pharmaceutical composition of claim 33, wherein said unit dosage form comprises 360 mg of dipyridamole.

38. The pharmaceutical composition of claim 33, wherein 50% to 80% of said dipyridamole is formulated for controlled release and 20% to 50% of said dipyridamole is formulated for immediate release.

39. The pharmaceutical composition of claim 28, wherein said unit dosage form further comprises 0.75 to 2.5 mg of prednisolone or an equivalent, equipotent amount of another corticosteroid, formulated for immediate release.

40. The pharmaceutical composition of claim 28, wherein said unit dosage form further comprises 0.75 to 2.5 mg of prednisolone or an equivalent, equipotent amount of another corticosteroid, formulated for controlled release.

41. The pharmaceutical composition of claim 39, comprising 1.8 mg of prednisolone or an equivalent, equipotent amount of another corticosteroid.

42. The pharmaceutical composition of claim 40, comprising 0.9 mg of prednisolone or an equivalent, equipotent amount of another corticosteroid.

43. The pharmaceutical composition of claim 39, wherein said corticosteroid is selected from prednisolone, prednisone, budesonide, methylprednisolone, fluticasone, betamethasone, and deflazacort.

44. The pharmaceutical composition of claim 39, wherein said corticosteroid is formulated as a coated non-pareil bead.

45. The pharmaceutical composition of claim 28, wherein said unit dosage form further comprises 0.75 to 3.75 mg of prednisolone, wherein 50% to 80% of said prednisolone is formulated for immediate release and 20% to 50% of said prednisolone is formulated for controlled release.

46. The pharmaceutical composition of claim 45, wherein said unit dosage form comprises an inner core comprising prednisolone formulated for controlled release and an outer coating comprising prednisolone formulated for immediate release.

47. The pharmaceutical composition of claim 46, wherein said inner core comprising 0.9 mg of prednisolone formulated for controlled release and an outer coating comprising 1.8 mg of prednisolone formulated for immediate release.

48. The pharmaceutical composition of claim 46, wherein said inner core comprising 0.45 mg of prednisolone formulated for controlled release and an outer coating comprising 0.9 mg of prednisolone formulated for immediate release.

49. A pharmaceutical composition in unit dosage form comprising 40 to 400 mg of dipyridamole formulated for controlled release and 0.75 to 3.75 mg of prednisolone or an equivalent, equipotent amount of another corticosteroid, formulated for controlled release or immediate release.

50. The pharmaceutical composition of claim 49, wherein said unit dosage form further comprises dipyridamole formulated for immediate release.

51. The pharmaceutical composition of claim 50, wherein 50% to 80% of said dipyridamole is formulated for controlled release and 20% to 50% of said dipyridamole is formulated for immediate release.

52. The pharmaceutical composition of claim 49, wherein said unit dosage form further comprises prednisolone or an equivalent, equipotent amount of another corticosteroid, formulated for controlled release and immediate release.

53. The pharmaceutical composition of claim 52, wherein 50% to 80% of said prednisolone or an equivalent, equipotent amount of another corticosteroid, is formulated for immediate release and 20% to 50% of said prednisolone or an equivalent, equipotent amount of another corticosteroid, is formulated for controlled release.

54. The pharmaceutical composition of claim 36, wherein said corticosteroid is formulated in a unit dosage form having a dissolution release profile under in vitro conditions in which at least 50% of the corticosteroid is released within the first 30 minutes of testing, wherein said in vitro conditions employ USP Dissolution Apparatus No. 1 at 37° C.±0.5° C. and 100 rpm in 0.1N HCl as dissolution medium for the first two hours, and a pH 6.8 phosphate buffer as the medium thereafter.

55. The pharmaceutical composition of claim 28, wherein said dipyridamole is formulated in a unit dosage form having a dissolution release profile under in vitro conditions in which at least 10-55% of the dipyridamole is released within the first two hours of testing and not less than 80% of the dipyridamole is released within 8 hours, wherein said in vitro conditions employ USP Dissolution Apparatus No. 1 at 37° C.±0.5° C. and 100 rpm in 0.1N HCl as dissolution medium for the first two hours, and a pH 6.8 phosphate buffer with 0.25% sodium lauryl sulfate as the medium thereafter.

56. The pharmaceutical composition of claim 28, wherein said dipyridamole is formulated in a unit dosage form having, upon administration to fed patients, an absorption rate constant of from 0.20 to 0.90 l/hr.

57. A kit comprising (i) the pharmaceutical composition in unit dosage form of claim 28; and (ii) instructions for administering the pharmaceutical composition for the treatment of an immunoinflammatory disease.

58. The kit of claim 57, further comprising instructions for administering said unit dosage form once or twice daily.
